(19) United States
(12) Patent Application Publication
(10) Pub. No.: US 2018/0028802 A1
(43) Pub. Date: Feb. 1, 2018

(54) DRY ELECTRODE

(71) Applicant: y-Brain Inc, Daejeon (KR)

(72) Inventors: Ki Won LEE, Seongnam-si (KR); Jong Min JANG, Suwon-si (KR); Sang Min LEE, Daegu (KR)

(21) Appl. No.: 15/220,358

(22) Filed: Jul. 26, 2016

(51) Int. Cl. A61N 1/04

(52) U.S. Cl. CPC A61N 1/0492; A61N 1/0496

(57) ABSTRACT

Disclosed is a dry electrode that electrically stimulates while contacting skin of a user, the dry electrode including a main body including a conductive body, and a nonconductive cover that surrounds the conductive body such that an area of the conductive body is exposed, and a patch detachably mounted on an area of the main body, in which the conductive body is exposed, one surface of the patch contacting the conductive body and an opposite surface of the patch contacting the skin of the user, the patch being configured to receive a current from the main body to electrically stimulate the skin of the user.

### BACKGROUND

The inventive concept relates to a dry electrode, and more particularly to a dry electrode that contacts skin of a user to electrically stimulate the skin of the user.

A low frequency treatment refers to using a low frequency for treatment, for example, of nerves or muscles by allowing a low frequency pulse to flow unto the nerves or muscles, and in this regard, studies on an electrode that transfers a low frequency pulse to a living body have been considerably established. Currently, low frequency treatment devices are being widely used, and products that may be used by the public as well as the medical professionals are being released.

Meanwhile, a brain electrical stimulation technology using a transcranial direct current stimulation (tDCS) is a stimulation technology that applies a current to the brain, and is known to be effective to improve a recognition ability and treat mental diseases such as depressions or attention deficit hyperactivity disorders (ADHD).

Accordingly, if the brain electrical stimulation technology may be used in everyday lives, the brain function may be improved, and mental diseases may be continuously treated by activating or restraining connections between nerves. However, in contrast to the low frequency stimulation apparatus, the electrical stimulation apparatus using tDCS needs to secure a high level of safety as the head is stimulated.

### PRIOR TECHNICAL DOCUMENTS

### Patent Documents

(Patent Document 1) Korean Patent Application Publication No. 10-2003-0045730

In particular, a proper electrode is not used in the transcranial direct current stimulation, current density on the electrode may become uneven. In detail, due to an edge effect of the electrode, a current density at a peripheral portion of the electrode may increase. Accordingly, as a high current may flow to a specific location of the electrode, the skin of the user may be burned under circumstances. In addition, sometime, the patch attached to the electrode may be delaminated or the patch may be thermally deformed.

### SUMMARY

The inventive concept has been made in an effort to solve the aforementioned problems, and provides a dry electrode that can prevent a current density at a peripheral portion of an electrode from increasing, by providing a patch with a hydrogel layer that has high impedance.

The inventive concept also provides a dry electrode that can prevent an over-potential from occurring between skin and an electrode by providing a hydrogel layer for a patch containing chorine ions and thus providing common ions between the skin and the electrode.

The inventive concept also provides a dry electrode that has a structure that may be adhered to skin.

The technical objects of the inventive concept are not limited to the above-mentioned one, and the other unmentioned technical objects will become apparent to those skilled ones in the art from the following description.

In accordance with an aspect of the inventive concept, there is provided a dry electrode that electrically stimulates while contacting skin of a user, the dry electrode including a main body including a conductive body, and a nonconductive cover that surrounds the conductive body such that an area of the conductive body is exposed, and a patch detachably mounted on the area of the conductive body that is exposed, one surface of the patch contacting the conductive body and an opposite surface of the patch contacting the skin of the user, the patch being configured to receive a current from the main body to electrically stimulate the skin of the user, wherein the patch includes a first hydrogel layer contacting the conductive body, and a second hydrogel layer having an impedance that is higher than that of the first hydrogel layer and contacting the skin of the user, and wherein a surface of the conductive body, which contacts the patch, has a shape that is deflected along one direction as a whole.

In accordance with another aspect of the inventive concept, there is provided an electrical stimulation apparatus including a frame mountable on a user, and a dry electrode formed on one surface of the frame, the dry electrode being configured to electrically stimulate skin of the user while contacting the skin of the user when the frame is mounted on the user, wherein the dry electrode includes a main body including a conductive body, and a nonconductive cover that surrounds the conductive body such that an area of the conductive body is exposed, and a patch detachably mounted on the area of the conductive body that is exposed, one surface of the patch contacting the conductive body and an opposite surface of the patch contacting the skin of the user, the patch being configured to receive a current from the main body to electrically stimulate the skin of the user, wherein the patch includes a first hydrogel layer that contacts the conductive body, and a second hydrogel layer having an impedance that is higher than that of the first hydrogel layer and contacting the skin of the user, and wherein a surface of the conductive body, which contacts the patch, has a shape that is deflected along one direction as a whole.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a perspective view of a dry electrode according to an embodiment of the inventive concept;

FIG. 2 is an exploded perspective view of a dry electrode of FIG. 1;

FIG. 3 is a sectional view of the dry electrode of FIG. 1 taken along direction x;

FIG. 4 is a sectional view of the dry electrode of FIG. 1 taken along direction y;

FIG. 5 is a sectional view of a dry electrode according to a modified embodiment of the inventive concept taken along direction x or direction y;

FIG. 6 is a bottom view of a conductive body of FIG. 2;

FIG. 7 is a sectional view taken along direction y for explaining a hinge function of the dry electrode of FIG. 1;

FIGS. 8 and 9 is a sectional view for explaining a method for coupling the main body and a patch of FIG. 1;

FIG. 10 is a perspective view of a dry electrode according to another embodiment of the inventive concept;

FIG. 11 is an electrical stimulation apparatus including a dry electrode according to embodiments of the inventive concept;

FIG. 12 is a perspective view of a dry electrode according to another embodiment of the inventive concept;

FIG. 13 is an exploded perspective view of the dry electrode of FIG. 12;

FIG. 14 is an exploded sectional view of the dry electrode of FIG. 12 taken along direction x;

FIG. 15 is a sectional view of the dry electrode of FIG. 12 taken along direction x; and

FIG. 16 is a sectional view of the dry electrode of FIG. 12 taken along direction y.

### DETAILED DESCRIPTION

Hereinafter, exemplary embodiments of the inventive concept will be described in detail with reference to the accompanying drawings. The above and other aspects, features and advantages of the invention will become apparent from the following description of the following embodiments given in conjunction with the accompanying drawings. However, the inventive concept is not limited to the embodiments disclosed below, but may be implemented in various forms. The embodiments of the inventive concept is provided to make the disclosure of the inventive concept complete and fully inform those skilled in the art to which the inventive concept pertains of the scope of the inventive concept. The same reference numerals denote the same elements throughout the specification.

Unless otherwise defined, all terms (including technical and scientific terms) used herein have the same meaning as commonly understood by those skilled in the art to which the inventive concept pertains. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein.

The terms used herein are provided to describe the embodiments but not to limit the inventive concept. In the specification, the singular forms include plural forms unless particularly mentioned. The terms “comprises” and/or “comprising” used herein does not exclude presence or addition of one or more other elements, in addition to the aforementioned elements.

Hereinafter, a dry electrode according to various embodiments of the inventive concept will be described with reference to the accompanying drawings.

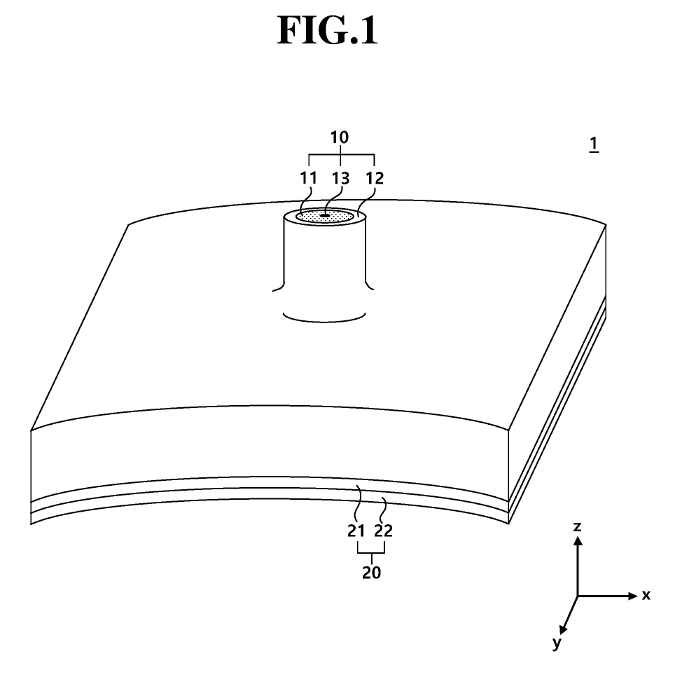

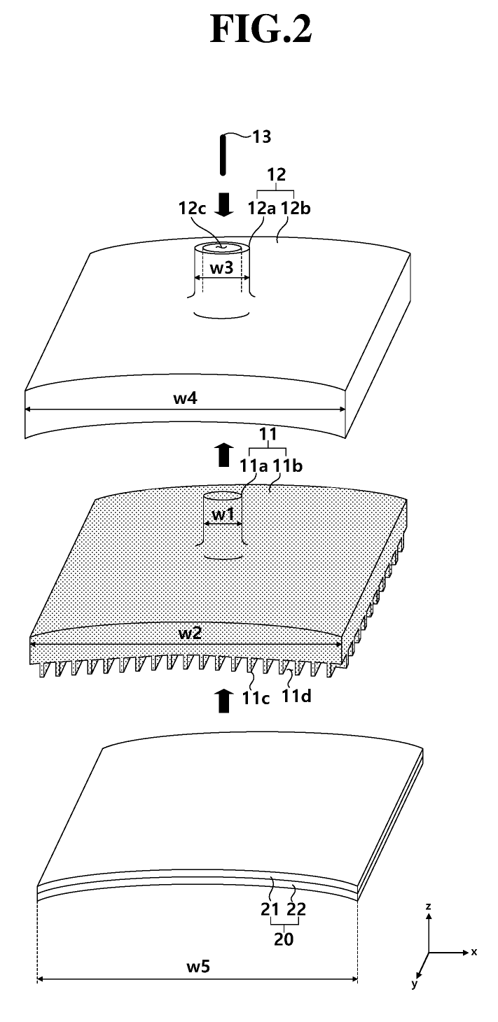

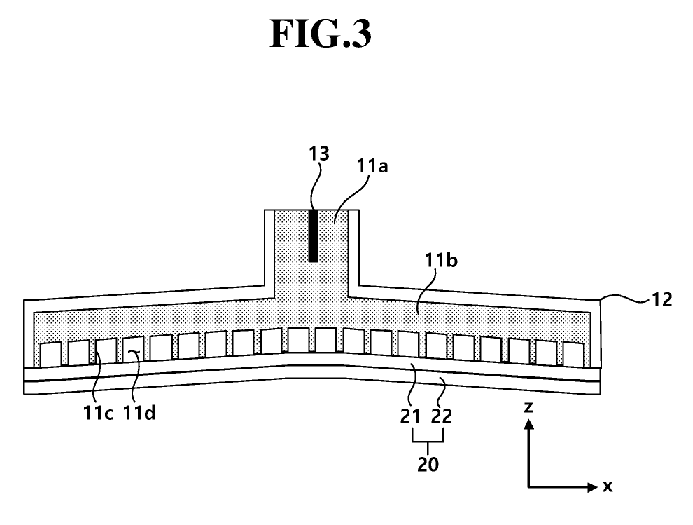

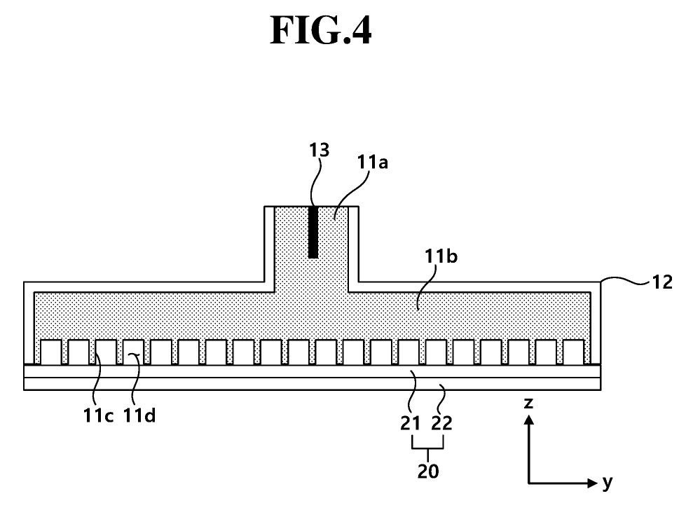

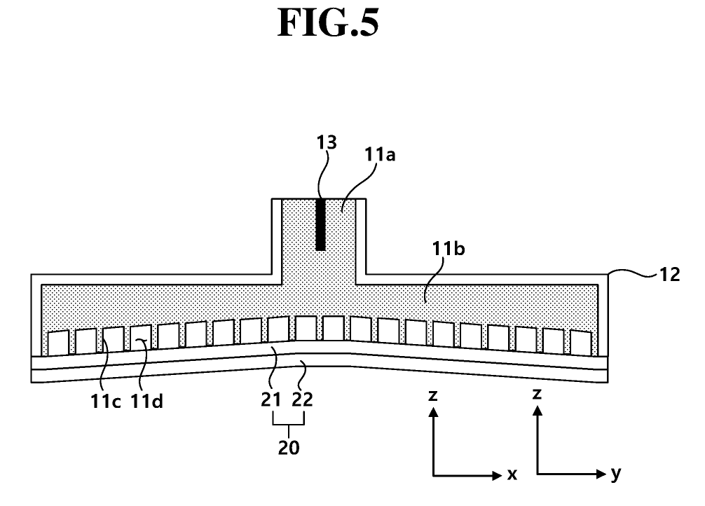

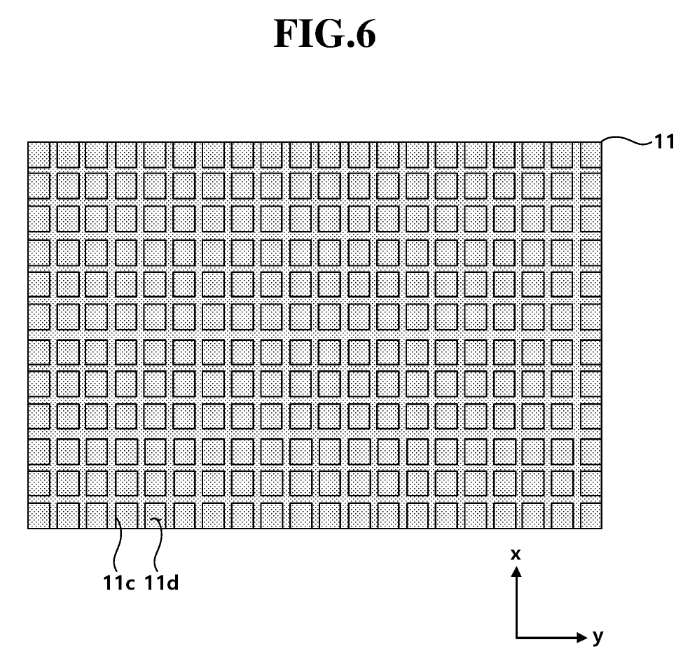

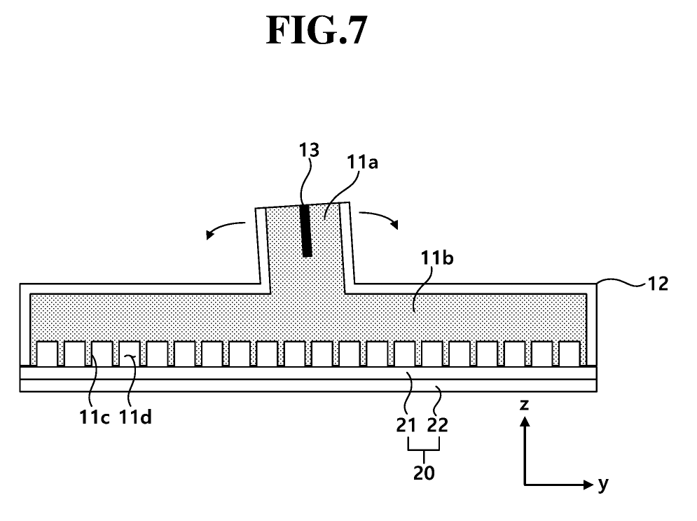

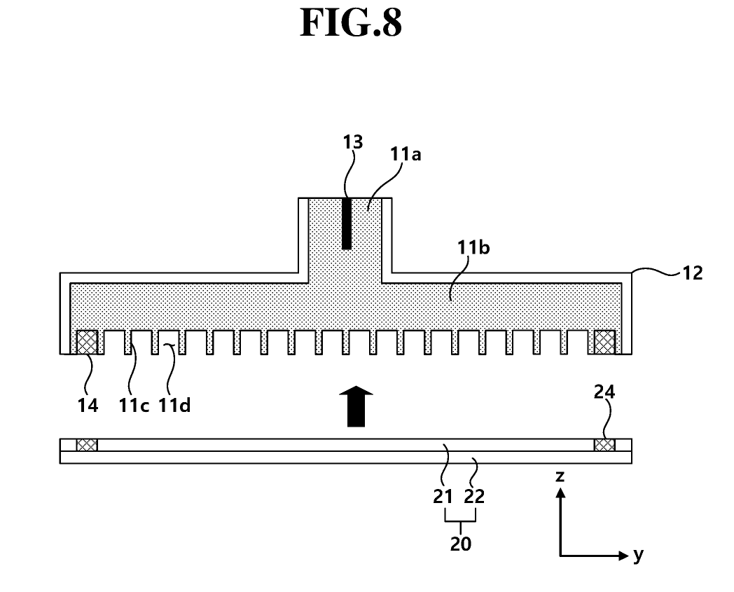

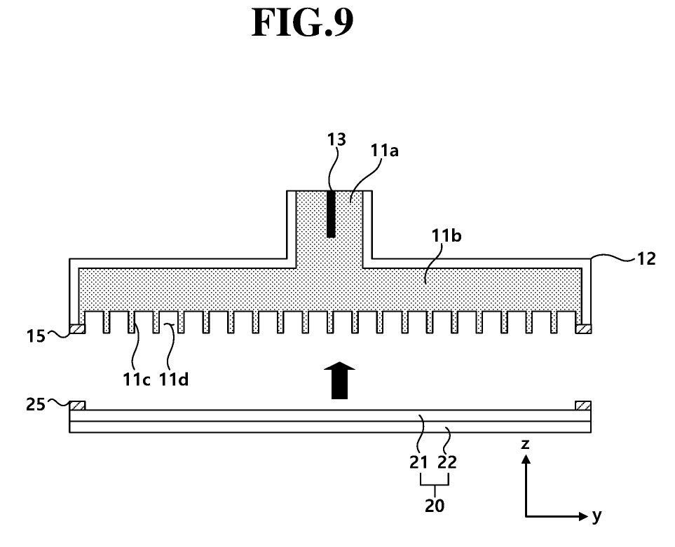

The dry electrode 1 according to an embodiment of the inventive concept will be described in the following with reference to FIGS. 1 to 9. FIG. 1 is a perspective view of a dry electrode 1 according to an embodiment of the inventive concept, FIG. 2 is an exploded perspective view of the dry electrode 1 of FIG. 1, FIG. 3 is a sectional view of the dry electrode 1 of FIG. 1 taken along direction x, FIG. 4 is a sectional view of the dry electrode 1 of FIG. 1 taken along direction y, FIG. 5 is a sectional view of a dry electrode according to a modified embodiment of the inventive concept taken along direction x or direction y, FIG. 6 is a bottom view of a conductive body 11 of FIG. 2; FIG. 7 is a sectional view for explaining a hinge function of the dry electrode 1 of FIG. 1 taken along direction y, and FIGS. 8 and 9 is a sectional view for explaining a method for coupling a main body 10 and a patch 20 of FIG. 1.

The dry electrode 1 according to an embodiment of the inventive concept may electrically stimulate skin of the user while contact it. For example, a current may be supplied to the dry electrode 1 through a conductive wire 13 extending from the outside of the dry electrode 1 to the inside of the dry electrode 1. Further, because the dry electrode 1 may in contact with the skin of the user, a current may be transferred to the skin of the user through the dry electrode 1 so that the user may be electrically stimulated.

Referring to FIGS. 1 and 2, the dry electrode 1 may include a main body 10 and a patch 20 that may be attached to and detached from the main body 10. Further, the main body 10 may include a conductive body 11, a nonconductive cover 12, and a conductive wire 13, and the patch 20 may include first and second hydrogel layers 21 and 22. However, the elements of FIGS. 1 and 2 are not essential, and thus a dry electrode 1 having a larger number of elements or a smaller number of elements may be implemented.

First, the main body 10 will be described. The main body 10 may include a conductive body 11 and a nonconductive cover 12 that surrounds the conductive body 11 such that an area of the conductive body 11 is exposed. If necessary, the conductive wire 13 may be connected to the conductive body 11.

Referring to FIG. 2, the conductive body 11 may include a first area 11a having a first width w1 and a second area 11b having a second width w2 that is larger than the first width w1. Further, the conductive body 11, for example, may have a shape in which the first area 11a is formed on the second area 11b and the first area 11a may protrude from the second area 11b along a third direction (direction z), but the inventive concept is not limited thereto. Further, the shape of the first area 11a is not limited to a cylindrical shape.

Referring to FIGS. 1, 3, and 4, the conductive wire 13 may be connected to the first area 11a of the conductive body 11. Accordingly, a current provided from the conductive wire 13 may flow to the second area 11b of the conductive body 11 via the first area 11a of the conductive body 11. That is, because a current flows from the first area 11a having a relatively small width to the second area 11b having a relatively large width, an electrical stimulus may be applied to skin of the user by using an area that is as wide as the area of a section of the second area 11b on the x-y plane. Accordingly, according to the dry electrode 1 according to the present embodiment, an electrical stimulus may be easily applied to a wide area.

In detail, the second area 11b of the conductive body 11 may have a surface that is bonded to the patch 20, and may be easily deformed due to flexible characteristics to help the patch 20 be adhered to the skin. Further, the first area 11a of the conductive body 11 may be an area that supports the second area 11b that is a deformed area and transfers a current to the conductive body 11.

Here, the second width w2 of the conductive body 11 may be smaller than a width w5 of the patch 20. That is, the cross-sectional area of the conductive body 11 on the x-y plane may be smaller than the cross-sectional area of the patch 20 on the x-y plane. According to the structural characteristics, a bonding surface of the second area 11b of the conductive body 11 may be completely covered by the patch 20 when the patch 20 is bonded to the second area 11b of the conductive body 11. Accordingly, because the second area 11b of the conductive body 11 is covered by the patch 20, the patch 20 may contact skin of the user when the dry electrode 1 contacts the skin of the user, and because the conductive body 11 may be prevented from directly contacting the skin of the user, a safety of the user who uses the dry electrode 1 may be secured.

Further, referring to FIGS. 2 to 4, the second area 11b of the conductive body 11 may have a rectangular parallelepiped shape as a whole, and for example, may have a deflected (bent) shape along a first direction (direction x). That is, the second area 11b of the conductive body 11 may have a shape that is convex towards the first area 11a, but the inventive concept is not limited thereto. In detail, referring to the sectional view taken along the first direction (direction x) of FIG. 3, it may be identified that the conductive body 11 is deflected (bent) along the first direction (direction x) when the conductive body 11 has a shape that is deflected (bent) along the first direction (direction x), and referring to the sectional view according to the second direction (direction y) of FIG. 4, it may be identified that the conductive body 11 is not defected along the second direction (direction y).

The dry electrode 1 may be used for an electrical stimulation apparatus that applies an electrical stimulus, for example, to a head, and because the conductive body 11 is curved along the first direction (direction x) as a whole, the dry electrode 1 may be easily adhered to the head in consideration of the shape of the head of a person is not flat but curved. Accordingly, the dry electrode 1 according to an embodiment of the inventive concept may have an excellent adherence property.

However, the shape of the conductive body 11 is not limited thereto, and in some embodiments, the second area 11b of the conductive body 11 may not be a deflected (curved) along the first direction (direction x) as a whole but a surface of the conductive body 11, which contacts the patch 20, may be deflected (curved), for example, along the first direction (direction x).

Further, in some embodiments, a central portion of a surface of the conductive body 11, which contacts the patch 20, may be concave such that the dry electrode 1 may be adhered to the forehead of a person. For example, referring to FIG. 5, it may be identified that a surface of the conductive body 11, which contacts the patch, may be deflected (curved) along the first direction (direction x) and the second direction (direction y). Through this, it may be seen that a central portion of a surface of the conductive body 11, which contacts the patch 20 has a depressed shape. Further, referring to FIGS. 2 to 6, a plurality of protrusions 11c may be formed in an area of the conductive body 11, which contacts the patch 20, that is, in an area of the second area 11b of the conductive body 11, which contacts the patch 20. The protrusions 11c may protrude from the second area 11b of the conductive body 11 along a third direction (direction z). Although lattice type protrusions are illustrated as the protrusions 11c in the drawings, the inventive concept is not limited thereto, but in some embodiments, the protrusions 11c may be replaced by protrusions of various shapes, such as straight protrusions, rectangular protrusions, honey-combed protrusions, spike-shaped protrusions, and spiral protrusions.

Grooves 11d may be formed between the protrusions 11c. A plurality of grooves 11d may be regularly formed in an area of the conductive body 11, which contacts the patch 20, that is, in an area of the second area 11b of the conductive body 11, which contacts the patch 20. For example, referring to FIG. 6, the grooves 11d may have a tetragonal shape, but the inventive concept is not limited thereto. However, in some embodiments, the grooves 11d may be irregularly formed, but the depth and sectional shape of the grooves 11d may be various.

Because the protrusions 11c may act as cushions or impact absorbers when the dry electrode 1 is adhered to skin of the user to contact the skin of the user, the dry electrode 1 according to an embodiment of the inventive concept may be adhered to the head with an excellent adherence performance.

Meanwhile, because the conductive body 11 is conductive and one end of the conductive body 11 is connected to the conductive wire 13, the conductive body 11 may receive a current from the conductive wire 13 to transfer the current to the patch 20.

Further, the conductive body 11 may be flexible, the shape of the conductive body 11 may be partially changed if necessary. For example, referring to FIG. 7, because an angle between the first area 11a and the second area 11b changed by using flexible characteristics of the conductive body 11, the conductive body 11 may have a hinge function. However, the dry electrode 1 may have a hinge function in other methods than the method of using the flexible characteristics of the conductive body 11. Accordingly, by using the dry electrode 1 according to the embodiment of the inventive concept, the dry electrode 1 may be easily adhered to the skin of the user through changing the angle or the shape of the dry electrode 1.

The conductive body 11 may include conductive silicon or flexible silicon to secure the conductivity and flexible characteristics to secure the conductivity and flexible characteristics, but the material of the conductive body 11 is not limited thereto.

The nonconductive cover 12 may surround the conductive body 11 such that an area of the conductive body 11 is exposed. In detail, referring to FIGS. 2 to 4, almost all the areas of the conductive body 11, except for a bottom surface of the second area 11b having the protrusions 11c and a top surface of the first area 11a connected to the conductive wire 13, may be covered by the nonconductive cover 12. Because the nonconductive cover 12 is formed of a nonconductive material and surrounds an outer surface of the dry electrode 1, an area of the dry electrode 1, which may contact a hand of the user, may be insulated. Accordingly, the user may safely use the dry electrode 1 by using the dry electrode 1 according to the embodiment of the inventive concept.

In detail, referring to FIGS. 2 to 4, the nonconductive cover 12 may include a third area 12a having a third width w3 and a fourth area 12b having a fourth width w3 that is larger than the third width w3. Further, the nonconductive cover 12, for example, may have a shape in which the third area 12a is formed on the fourth area 12b and the third area 12a may protrude from the fourth area 12b along the third direction (direction z), but the inventive concept is not limited thereto. Further, the shape of the third area 12a is not limited to a cylindrical shape.

Meanwhile, the conductive body 11 may be coupled to the nonconductive cover 12. To achieve this, because an empty space, to which the conductive body 11 may be coupled, may be formed in the interior of the nonconductive cover 12, the conductive body 11 may be inserted into the nonconductive cover 12. For example, a hole 12c may be formed in the third area 12a of the nonconductive cover 12, and an empty space also may be formed in the fourth area 12b of the nonconductive cover 12. Accordingly, the first area 11a of the conductive body 11 may be inserted into the third area 12a of the nonconductive cover 12, the second area 11b of the conductive body 11 may be inserted into the fourth area 12b of the nonconductive cover 12, and the protrusions 11c of the conductive body 11 may be exposed. To achieve this, the first width w1 of the first area 11a may be made to be narrower than the third width w3 of the third area 12a, and the second width w2 of the second area 11b may be made to be narrower than the fourth width w4 of the fourth area 12b.

In addition, the conductive wire 13 may extend from the outside of the dry electrode 1 to the inside of the dry electrode 1, and for example, may contact a portion of the conductive body 11.

Next, the patch 20 will be described. The patch 20 may be attached to and detached from an area of the main body 10, to which the conductive body 11 is exposed, and one surface of the patch 20 may contact the conductive body 11 and an opposite surface of the patch 20 may contact skin of the user. Further, the patch 20 may receive a current from the main body 10 to electrically stimulate the skin of the user.

In detail, referring to FIGS. 1 to 4, the patch 20 may be attached to and detached from a bottom surface of the main body 10, that is, a surface of the conductive body 11, on which a lattice type structure is formed. Although a bonding layer (not illustrated) may be formed, for example, on one surface of the patch 20 for attachment and detachment of the patch 20, the method of attaching and detaching the patch 20 is not limited thereto but various methods may be used.

For example, the first hydrogel layer 21 of the patch 20 may contact the conductive body 11, and the first hydrogel layer 21 has a high bonding force of not less than 200 gf/cm. Accordingly, because the first hydrogel layer 21 is attached to one surface of the second hydrogel layer 22 and another surface of the second hydrogel layer 22 is bonded to the conductive body 11, the patch 20 may be attached to the conductive body 11, and the patch 20 may be separated from the conductive body 11 if necessary.

In addition, referring to FIG. 8, magnets 14 and 24 may be formed in at least one of the main body 10 and the patch 20 so that the patch 20 may be attached to and detached from the main body 10 through a magnetic force. Further, referring to FIG. 9, coupling structures 15 and 25 may be formed in at least one of the main body 10 and the patch 20 so that the patch 20 may be attached to and detached from the main body 10 through coupling of the coupling structures 15 and 25. The coupling structures 15 and 25 may be structures for male/female coupling, such as snap buttons (press buttons) or may be structures for mechanical coupling, such as latches, but the inventive concept is not limited thereto.

In this way, according to the dry electrode 1 according to the embodiment of the inventive concept, because the patch 20 may be attached and detached, it may be easily replaced when it is deformed or worn.

Meanwhile, the patch 20 may include first and second hydrogel layers 21 and 22.

In detail, the first hydrogel layer 21 may contact the conductive body 11, and in more detail, may contact the protrusions 11c. Accordingly, the first hydrogel layer 21 may transfer a current provided from the conductive body 11 to the second hydrogel layer 22, and one surface of the patch 20, which contacts the conductive body 11, may be one surface of the first hydrogel layer 21.

A bonding force of the second hydrogel layer 22 is as low as not more than 200 gf/cm. Accordingly, the second hydrogel layer 22 may not be stuck to skin of the user. Accordingly, the dry electrode 1 may be used for a wearable device that is repeatedly mounted on and dismounted from the user, and in detail, may be used for a transcranial current stimulation apparatus.

Further, the second hydrogel layer 22 may contact skin of the user while having an impedance that is higher than that of the first hydrogel layer 21. For example, a transcranial direct current stimulation is performed by using the dry electrode 1, the second hydrogel layer 22 may have an impedance of not less than 4 kΩ. Accordingly, according to the dry electrode 1 according to the embodiment of the inventive concept, because the second hydrogel layer 22 has a high impedance, a current density of a peripheral portion of the electrode may be prevented from increasing due to an edge effect of the electrode. Accordingly, because a constant current density may be maintained in an entire area of the patch 20, an electrical stimulus may be stably applied through the dry electrode 1.

Further, skin includes keratin having a relatively high impedance and other parts having relatively low impedances, and because a current may easily flow through the other parts of the skin having low impedances, the current may be concentrated on an area while flowing. However, according to the dry electrode 1 according to the embodiment of the inventive concept, because an influence by an impedance difference between the keratin and the other parts may be decreased by the second hydrogel layer 22 of a high impedance, a current may be prevented from being concentrated on a portion of the skin while flowing and a uniform current density may be formed in an entire area of skin, which contacts the dry electrode 1. Accordingly, an electrical stimulus may be stably applied through the dry electrode 1.

Further, the second hydrogel layer 22 may include chlorine ions. Because skin of the user also include chlorine ions, the skin of the user and the second hydrogel layer 22, which contacts the skin of the user include common icons. Through this, because an over-potential formed between the dry electrode 1 and the skin of the user may be lowered, a stimulus of an unnecessary magnitude may be prevented from being formed in the skin of the user. Accordingly, an electrical stimulus may be stably applied through the dry electrode 1. However, in some embodiments, the first hydrogel layer 21 as well as the second hydrogel layer 22 may include chlorine ions.

Meanwhile, because the first hydrogel layer 21 may function as a compressible cushion when the dry electrode 1 contacts skin of the user, the dry electrode 1 may be adhered to the skin of the user.

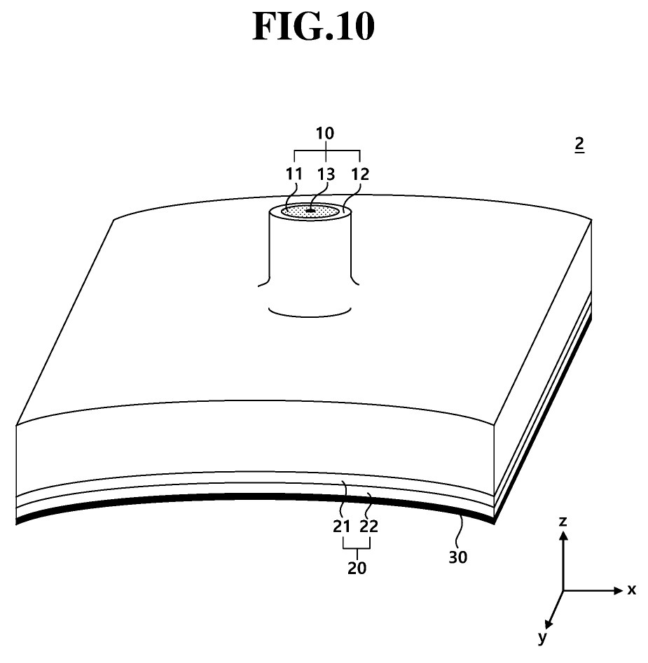

The dry electrode 2 according to an embodiment of the inventive concept will be described in the following with reference to FIG. 10. However, a difference from the dry electrode 1 according to an embodiment of the inventive concept will be mainly described. Referring to FIG. 10, a perspective view of a dry electrode 2 according to another embodiment of the inventive concept is illustrated.

Referring to FIG. 10, the dry electrode 2 may further include a delamination layer 30 that covers the second hydrogel layer 22 of the patch 20. The delamination layer 30 is a cover for protecting the patch 20, and may protect the patch 20 before use of the dry electrode 2. Accordingly, the delamination layer 30 may be removed during use of the dry electrode 2, and the second hydrogel layer 22 may contact skin of the user.

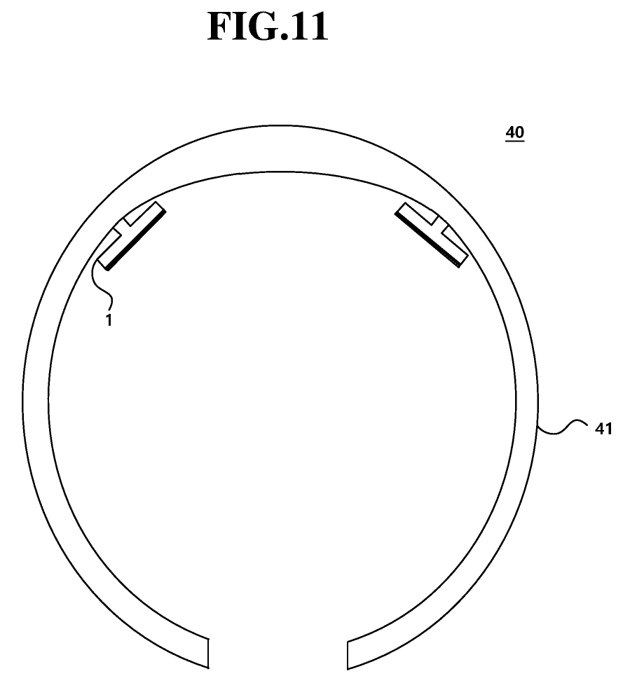

Hereinafter, an electrical stimulation apparatus 40 including the dry electrode 1 according to embodiments of the inventive concept will be described with reference to FIG. 11. Hereinafter, referring to FIG. 11, the electrical stimulation apparatus 40 including the dry electrode 1 according to embodiments of the inventive concept is illustrated.

Referring to FIG. 11, the electrical stimulation apparatus 40 may include a frame 41 and a dry electrode 1 attached to the frame 41. The dry electrode 1 may be attached to an inner surface of the frame 41, and it may contact the head of the user when the electrical stimulation apparatus 40 is mounted on the head. Meanwhile, although the frame 41 of FIG. 11 has a shape that may be mounted on the head of the user, any shape that may be mounted on the user is possible while the inventive concept is not limited thereto, and thus the electrical stimulation apparatus 40 may be implemented by various wearable devices.

Because the outside of the dry electrode 1 is surrounded by the nonconductive cover 12, the user may safely use the dry electrode 1. Further, a portion of the dry electrode 1, which contacts the frame 41, has a hinge function by which an angle of the dry electrode 1 may be adjusted, the user may easily adhere the dry electrode 1 to his or her head.

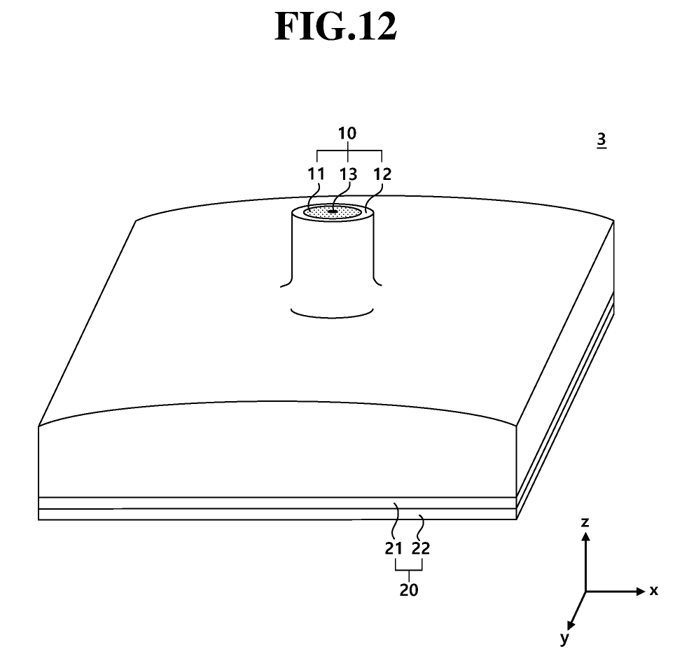

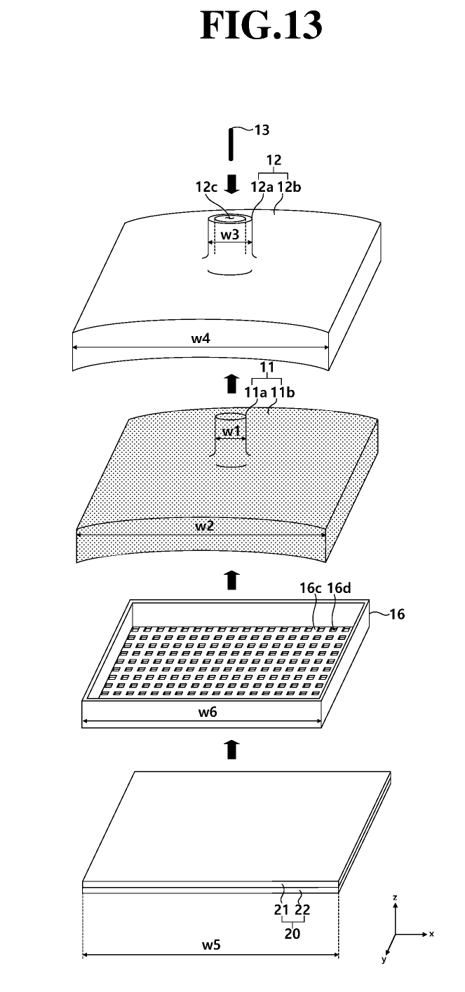

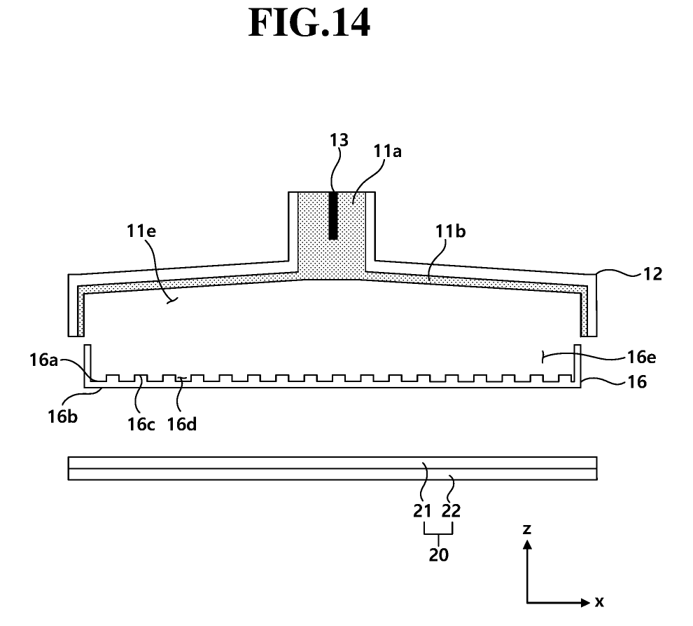

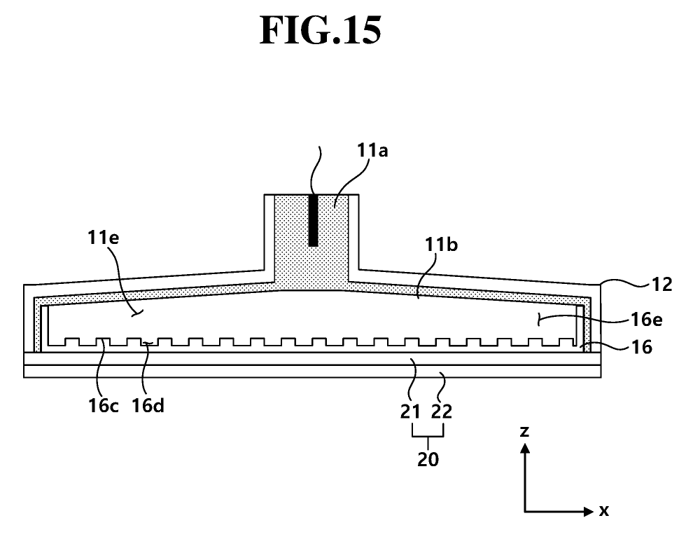

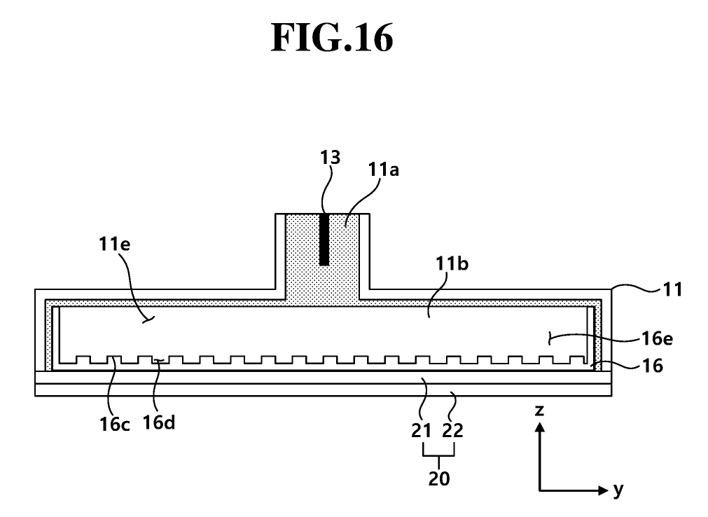

The dry electrode 3 according to another embodiment of the inventive concept will be described in the following with reference to FIGS. 12 to 16. However, a difference from the dry electrode 1 according to an embodiment of the inventive concept will be mainly described. Referring to FIG. 12, a perspective view of a dry electrode 3 according to another embodiment of the inventive concept is illustrated. FIG. 13 is an exploded perspective view of the dry electrode 3 of FIG. 12, FIG. 14 is an exploded sectional view of the dry electrode 3 of FIG. 12 taken along direction x, FIG. 15 is a sectional view of the dry electrode 3 of FIG. 12 taken along direction x, and FIG. 16 is a sectional view of the dry electrode 3 of FIG. 12 taken along direction y.

Referring to FIGS. 12 to 16, the dry electrode 3 according to the present embodiment may further include a second conductive body 16 in addition to a first conductive body 11, unlike the dry electrode 1 according to the previous embodiment of the inventive concept.

That is, referring to FIGS. 12 to 14, the main body 10 may include a first conductive body 11 one surface of which is opened and that has a first space 11e in the interior thereof, a nonconductive cover 12 that surrounds the first conductive body 11 such that the opened surface of the first conductive body 11 is exposed, and a second conductive body 16 that covers the opened surface of the first conductive body 11. Further, the second conductive body 16 may include a first surface 16a that contacts the first conductive body 11 and a second surface 16b that is opposite to the first surface 16a.

In detail, referring to FIGS. 12 to 14, the first space 11e may be formed in the interior of the first conductive body 11. The first space 11e is an empty space, and may be a space for increasing a degree of freedom by which the patch 20 may be deformed according to the head shape of the user when the dry electrode 3 contacts the head of the user. Because one surface (for example, a lower surface) of the first conductive body 11 may be opened, the first space 11e may be exposed through the opened lower surface.

Further, referring to FIGS. 13 to 16, the second conductive body 16 may include a first surface 16a and a second surface 16b, and the second surface 16b may be a surface that is opposite to the first surface 16a.

The second conductive body 16 may be formed to cover the opened surface of the first conductive body 11, and then the first surface 16a of the second conductive body 16 may fact the first conductive body 11. Referring to FIGS. 15 and 16, the first space 11e may be preserved while being surrounded by the first conductive body 11 and the first surface 16a of the second conductive body 16 even though the opened surface of the first body 11 is covered by the second conductive body 16. Accordingly, according to the dry electrode 3 according to the present embodiment, because a degree of freedom by which the patch 20 may be deformed according to the head shape of the user as the first space 11e of the main body 10 is preserved, the dry electrode 3 may be easily deformed due to the flexible characteristics, which may help the patch 20 to be adhered to skin.

As long as the second conductive body 16 may covers the opened surface of the first conductive body 11 while preserving the first space 11e of the first conductive body 11, the shape of the second conductive body 16 is not limited. However, referring to FIGS. 13 to 16, the second conductive body 16 may have a shape one surface (for example, an upper surface) of which is opened, and a second space 16e that is an empty space may be formed in the interior of the second conductive body 16. The second space 16e may be exposed through the opened upper space, and at least a portion of the second space 16e may be surrounded by the first surface 16a of the second conductive body 16. Because the second conductive body 16 includes the second space 16e, the first space 11e may be preserved by allowing the first space 11e and the second space 16e to overlap each other even though the opened surface of the first conductive body 11 is covered by the second conductive body 16.

In detail, referring to FIGS. 15 and 16, the second conductive body 16 may be inserted into the main body 10 by inserting the second conductive body 16 into the first space 11e of the first conductive body 11, but the coupling method is not limited thereto and various coupling forms such as protrusions may be used. When the second conductive body 16 is inserted into the first space 11e of the first conductive body 11, the second conductive body 16 may cover the opened surface of the first conductive body 11 and the opened surface of the second conductive body 16 may face the inner surface of the first conductive body 11. Accordingly, the first space 11e of the first conductive body 11 and the second space 16e of the second conductive body 16 may overlap each other, an empty space may be present in the main body 10.

Meanwhile, referring to FIGS. 13 to 16, a plurality of grooves 16d may be formed on the first surface 16a of the second conductive body 16. That is, the grooves 11d may be regularly formed on the first surface 16a that is opposite to the second surface 16b of the second conductive body 16, which contacts the patch 20. For example, although the groove 16d may have a tetragonal sectional shape such that a lattice structure is formed on the first surface 16a of the second conductive body 16, the inventive concept is not limited thereto. However, in some embodiments, the grooves 16d may be irregularly formed, but the depth and sectional shape of the grooves 16d may be various.

As the plurality of grooves 16d are formed on the first surface 16a of the second conductive body 16, a plurality of protrusions 16c may be formed on the first surface 16a, and for example, the protrusions 16c may protrude along a third direction (direction z). Although lattice type protrusions are illustrated as the protrusions 16c in the drawings, the inventive concept is not limited thereto, but in some embodiments, the protrusions 16c may be replaced by protrusions of various shapes, such as straight protrusions, rectangular protrusions, honey-combed protrusions, spike-shaped protrusions, and spiral protrusions.

As described above, because a thickness of the second conductive body 16 may decrease as the plurality of grooves 16d are formed on the first surface 16a of the second conductive body 16, it may help the patch 20 to be easily deformed according to the head shape of the user when the patch 20 is coupled to the second conductive body 16.

In addition, the first and second conductive bodies 11 and 16 may include conductive silicon or flexible silicon to secure the conductivity and flexible characteristics, but the materials of the first and second conductive body 11 and 16 are not limited thereto.

The patch 20 may be attached to and detached from the second surface 16b of the second conductive body 16, and may be a patch 20 one surface of which contacts the second surface 16b of the second conductive body 16 and an opposite surface of which contacts skin of the user. Further, the patch 20 may receive a current through the first and second conductive bodies 11 and 16 to electrically stimulate skin of the user, and the descriptions of the embodiments of the inventive concept may be applied.

Here, the second width w2 of the first conductive body 11 and the width w6 of the second surface 16b of the second conductive body 16 may be narrower than the width w5 of the patch 20. That is, the cross-sectional area of the first conductive body 11 on the x-y plane and the cross-sectional area of the second surface 16b of the second conductive body 16 on the x-y plane may be smaller than the cross-cross-sectional area of the patch on the x-y plane. Due to the structural characteristics, a bonding surface between the second area 11b of the first conductive body 11 and the second area 16b of the second conductive body 16 may be completely covered by the patch 20 when the patch 20 is bonded to the second area 11b of the first conductive body 11 and the second surface 16b of the second conductive body 16. Accordingly, because the patch 20 may come into contact with skin of the user and the first conductive body 11 and the second conductive body 16 may be prevented from coming into direct contact with the skin of the user when the dry electrode 1 contacts the skin of the user because the second area 11b of the first conductive body 11 and the second surface 16b of the second conductive body 16 are covered by the patch 20, the safety of the user who uses the dry electrode 3 can be secured.

According to the inventive concept, a current density at a peripheral portion of the electrode can be prevented from increasing by providing a hydrogel layer including a patch having a high impedance.

Further, according to the inventive concept, because a hydrogel layer for a patch contains chlorine ions, common ions are present between skin and an electrode so that an over voltage can be prevented from occurring between skin and the electrode.

Furthermore, according to the inventive concept, because the dry electrode has a structure that may be adhered to skin, an electrical stimulus may be stably applied to a designated location.

However, the effects of the inventive concept are not limited to the above-mentioned ones.

Although the exemplary embodiments of the inventive concept have been described with reference to the accompanying drawings, it will be understood by those skilled in the art to which the inventive concept pertains that the inventive concept can be carried out in other detailed forms without changing the technical spirits and essential features thereof. Therefore, the above-described embodiments are exemplary in all aspects, and should be construed not to be restrictive.

What is claimed is:

1. A dry electrode that electrically stimulates while contacting skin of a user, the dry electrode comprising:
a main body comprising a conductive body, and a nonconductive cover that surrounds the conductive body such that an area of the conductive body is exposed; and
a patch detachably mounted on the area of the conductive body that is exposed, one surface of the patch contacting the conductive body and an opposite surface of the patch contacting the skin of the user, the patch being configured to receive a current from the main body to electrically stimulate the skin of the user,
wherein the patch comprises:
a first hydrogel layer contacting the conductive body; and
a second hydrogel layer having an impedance that is higher than that of the first hydrogel layer and contacting the skin of the user, and
wherein a surface of the conductive body, which contacts the patch, has a shape that is deflected along one direction as a whole.

2. The dry electrode of claim 1, wherein a cross-sectional area of the conductive body in the one direction is smaller than a cross-sectional area of the patch in the one direction.

3. The dry electrode of claim 1, wherein the second hydrogel layer comprises chlorine ions.

4. The dry electrode of claim 1, wherein a plurality of protrusions are formed on a surface of the conductive body, which contacts the patch.

5. The dry electrode of claim 4, wherein the conductive body comprises:
a first area having a first width; and
a second area having a second width that is larger than the first width, and
wherein the first area is formed on the second area, and one of the protrusions is formed on a surface of the second area, which contacts the patch.

6. The dry electrode of claim 1, wherein the conductive body comprises conductive silicon and is flexible.

7. A dry electrode that electrically stimulates while contacting skin of a user, the dry electrode comprising:
a first conductive body one surface of which is opened and having a first space in the interior thereof, a nonconductive cover surrounding at least a portion of the first conductive body, and a main body comprises a second conductive body formed to cover the opened surface of the first conductive body, the second conductive body comprising a first surface that faces the first conductive body and a second surface that is opposite to the first surface; and
a patch detachably mounted on the second surface of the second conductive body of the main body, one surface of which contacts the second surface and an opposite surface of which contacts the skin of the user, the patch being configured to receive a current from the main body to electrically stimulate the skin of the user,
wherein the first space of the first conductive body is surrounded by the first conductive body and the first surface of the second conductive body.

8. The dry electrode of claim 7, wherein a cross-sectional area of the second surface of the second conductive body is smaller than a cross-sectional area of one surface of the patch.

9. The dry electrode of claim 7, wherein the second conductive body has an opened surface and has a second space in the interior thereof, the first surface of the second conductive body surrounds at least a portion of the second space of the second conductive body, the second conductive body is inserted into the first space of the first conductive body such that the second conductive body covers the opened surface of the first conductive body and the opened surface of the second conductive body faces an inner surface of the first conductive body, and the first space of the first conductive body and the second space of the second conductive body overlap each other.

10. The dry electrode of claim 7, wherein a plurality of grooves are formed on the first surface of the second conductive body.

11. The dry electrode of claim 7, wherein the patch comprises a first hydrogel layer contacting the second surface of the second conductive body, and a second hydrogel layer having an impedance that is higher than that of the first hydrogel layer, the second hydrogel layer contacting the skin of the user.

12. An electrical stimulation apparatus comprising:
a frame mountable on a user; and
a dry electrode formed on one surface of the frame, the dry electrode being configured to electrically stimulate skin of the user while contacting the skin of the user when the frame is mounted on the user,
wherein the dry electrode comprises:
a main body comprising a conductive body, and a nonconductive cover that surrounds the conductive body such that an area of the conductive body is exposed; and
a patch detachably mounted on the area of the conductive body that is exposed, one surface of the patch contacting the conductive body and an opposite surface of the patch contacting the skin of the user, the patch being configured to receive a current from the main body to electrically stimulate the skin of the user,
wherein the patch comprises:
a first hydrogel layer that contacts the conductive body; and
a second hydrogel layer having an impedance that is higher than that of the first hydrogel layer and contacting the skin of the user, and
wherein a surface of the conductive body, which contacts the patch, has a shape that is bent along one direction as a whole.

13. The electrical stimulation apparatus of claim 12, wherein a cross-sectional area of the conductive body in the one direction is smaller than a cross-sectional area of the patch in the one direction.

14. The electrical stimulation apparatus of claim 12, wherein the second hydrogel layer comprises chlorine ions.

15. The electrical stimulation apparatus of claim 12, wherein a plurality of protrusions are formed on a surface of the conductive body, which contacts the patch.

16. The electrical stimulation apparatus of claim 15, wherein the conductive body comprises:
a first area having a first width; and
a second area having a second width that is larger than the first width, and
wherein the first area is formed on the second area, and a protrusion is formed on a surface of the second area, which contacts the patch.

17. The electrical stimulation apparatus of claim 12, wherein the conductive body comprises conductive silicon and is flexible.
